(19) United States
(12) Patent Application Publication
(10) Pub. No.: US 2024/0188579 A1
(43) Pub. Date: Jun. 13, 2024

(54) WHEY PROTEIN-CONTAINING PRODUCT ENRICHED IN IMMUNOGLOBULINS

(71) Applicant: FrieslandCampina Nederland B.V., Amersfoort (NL)

(72) Inventors: Weiwei Li, Wageningen (NL); Chris Thérèse Emilienne Dotremont, Wageningen (NL); Christine Counet, Wageningen (NL); Alfred Willy Bonte, Wageningen (NL); Albert Bruno Verver, Wageningen (NL)

(21) Appl. No.: 18/287,587

(22) PCT Filed: Apr. 28, 2022
(86) PCT No.: PCT/EP2022/061383

§ 371 Date: Oct. 19, 2023

(30) Foreign Application Priority Data
Apr. 29, 2021 (EP) 21171274.0

(51) Int. Cl. A23C 9/142; A23C 9/146; A23C 21/00; A23J 1/20; A23L 33/00; A23L 33/19; B01D 15/38; C07K 1/34

(52) U.S. Cl. CPC A23C 9/1422; A23C 9/1465; A23C 21/00; A23J 1/205; A23L 33/19; A23L 33/40; B01D 15/3847; C07K 1/34

(57) ABSTRACT

Process for producing a whey protein-containing product enriched in immunoglobulins, said process comprising the steps of: (i) cross-flow filtration of casein-reduced milk using a membrane with a molecular weight cut-off (MWCO) of 500-1000 kDa, preferably 500-800 kDa, or a pore size of 50-100 nm, preferably 50-80 nm, thereby obtaining a permeate enriched in lactose, salts, α-lactalbumin and β-lactoglobulin, and an UF retentate, and (ii) subjecting said UF retentate to mixed mode chromatography, wherein immunoglobulins adhere to a resin and are subsequently eluted to form said whey protein-containing product enriched in immunoglobulins.

The present invention relates to a whey protein-containing product with a high immunoglobulin content, its production and use.

Milk provides the sole source of nutrition for mammalian offspring until they are able to digest food from other sources. Colostrum and milk of all lactating animals contain immunoglobulins (Ig's), which provide the offspring immunological protection against microbial pathogens and toxins and protect the mammary gland against infections. The major classes of immunoglobulins in bovine and human milk are IgG, IgA, and IgM, which differ in structure and biological activity. IgG can be subdivided in IgG₁ and IgG₂. In human milk, the major Ig is IgA. Human breast milk contains about 85-90 wt % IgA, about 2-3 wt % IgG, and about 8-10 wt % IgM (J. A. Cakebread et al., J. Agric. Food Chem., 63 (2015) 7311-7316). The total Ig concentration in human breast milk is about 1.5 times the Ig content of bovine milk (J. A. van Neerven et al., J. Allergy Clin. Immunol., October 2012, 853-858).

In bovine milk, on the other hand, the major Ig is IgG. Mature bovine milk contains about 80 wt % IgG (the far majority being IgG₁), about 10 wt % IgM, and about 10% IgA. The Ig-content of bovine colostrum is much higher than that of mature bovine milk: 70-80% of the total protein content of colostrum are Ig's, whereas in mature bovine milk they only provide for 1-2 wt % of the total protein content.

Infant formula is prepared by combining at least one source of whey protein, at least a source of milk (casein) proteins, at least one source of lipids, at least one carbohydrate source, vitamins, and minerals. Bovine milk is one of the applied sources for these proteins, carbohydrates, lipids, and vitamins.

There is a continuing desire to produce infant formula which resembles human breast milk as closely as possible. Hence, there is a desire to increase the active immunoglobulin content of infant formula in order to reach that goal.

Infant formula is generally prepared by combining milk with at least one source of whey proteins, at least one source of lipids, at least one carbohydrate source, and vitamins and minerals.

The whey protein source is preferably selected from whey protein concentrate (WPC) and serum protein concentrate (SPC). Both products are the result of separating skimmed milk into a casein-rich and a whey protein-rich fraction; either by renneting (i.e. cheese making), acidification, or microfiltration. Immunoglobulins mainly exist in the milk serum/whey phase, instead of the casein micelle phase, and are therefore considered whey proteins.

Whey protein concentrate (WPC) is conventionally obtained by ultrafiltration and/or reverse osmosis and optionally demineralization of acid or cheese whey. By ultrafiltration, a large part of the water, lactose and ash are removed from the product, thereby concentrating the whey proteins. Reverse osmosis can be used to remove water and to further concentrate the WPC. Serum protein concentrate (SPC) is also a concentrated whey protein product and differs from WPC in the origin of the whey fraction. Instead of acid or cheese whey, the whey proteins in SPC result from microfiltration of skimmed milk; they are generally referred to as native whey. Said microfiltration results in a concentrated casein retentate fraction and a serum fraction containing most of the whey proteins as the permeate fraction. Conventionally, this permeate fraction is then subjected to ultrafiltration and/or reverse osmosis in order to remove lactose, ash, and water.

The UF membrane used in these conventional processes has a molecular weight cut-off (MWCO) in the range 5-10 kDa. As a result, water, lactose, and minerals pass the membrane, whereas the proteins are concentrated in the retentate.

The immunoglobulin content of regular WPC and SPC is below 6 wt %, based on total whey protein. This is comparable to the content in mature bovine milk, which means that conventional WPC preparation does not lead to significant enrichment of Ig content.

EP 0 320 152 discloses a method for producing a WPC enriched in immunoglobulins, using two different processes.

In the first process, whey is subjected to ultrafiltration using a membrane with a molecular weight cut-off (MWCO) of 500 kDa.

In the second process, the whey is first subjected to anion exchange chromatography. In contrast to other whey proteins, most of the immunoglobulins are not bound by the anion exchange resin and thus form the effluent. This effluent is then subjected to ultrafiltration using a membrane with a MWCO of 500 kDa.

Ig-enrichment of WPC using anion exchange resins is also disclosed in WO 01/41584; example 4.

The disadvantage of these procedures is that anion exchange resins do not bind immunoglobulins, but instead the much more abundant other whey proteins. This leads to a high capacity demand of the chromatography process.

H. J. Heidebrecht et al., J. Chromatogr. A 1562 (2018) 59-68, discloses the isolation of bovine IgG from colostral bovine whey using mixed mode chromatography. Mixed mode chromatography is based on a combination of hydrophobic interactions and cation exchange.

Mixed mode chromatography resins bind immunoglobulins instead of most other whey proteins, meaning that lower chromatography capacity is required compared to anion exchange chromatography.

However, there is still a desire to further improve the chromatography capacity and to further improve the purity of the obtained immunoglobulins.

This object is met with the process of the present invention, which involves a cross-flow filtration step using a specific membrane prior to the chromatography step. This allows to further increase the binding capacity of the chromatography resin, i.e. the amount of immunoglobulins per resin volume per cycle. The prior filtration step reduces the number of whey proteins that may compete with immunoglobulins in binding to the resin, resulting in higher binding capacity and higher mass transfer of immunoglobulins to the resin.

The present invention therefore relates to the provision of a whey protein-containing product with a higher immunoglobulin content than regular whey protein-containing products (such as WPC and SPC) and to a process resulting in an enrichment of total Ig content, relative to total protein, of a factor of at least 10, preferably at least 15, most preferably at least 20. The whey protein-containing product according to the present invention contains at least 40 wt %, preferably 50-80 wt % IgG, based on total protein. The total protein content of the whey protein-containing product is determined by the BCA method, as described in R. A. Brown et al., Analytical Biochemistry, volume 180 (1), July 1989, 136-139. This IgG content is determined using the RP-HPLC-based method can be used, as described by D. F. Elgar et al., Journal of Chromatogeraphy A, Volume 878, 2000, 183-196).

The whey protein-containing product is prepared by a process comprising the steps of:

(i) cross-flow filtration of casein-reduced milk using a membrane with a molecular weight cut-off (MWCO) of 500-1000 kDa, preferably 500-800 kDa, or a pore size of 50-100 nm, preferably 50-80 nm, thereby obtaining a permeate enriched in lactose, salts, α-lactalbumin and β-lactoglobulin, and an UF retentate, and

(ii) subjecting said UF retentate to mixed mode chromatography, wherein immunoglobulins adhere to a resin and are subsequently eluted to form said whey protein-containing product enriched in immunoglobulins.

The term casein-reduced milk refers to any milk fraction that has been subjected to a process that reduces casein, such as casein precipitation (resulting in acid whey), a cheese-making process (resulting in cheese whey), or microfiltration so separate casein and serum proteins (resulting in native whey). The casein-reduced milk therefore preferably relates to acid whey, cheese whey, or native whey. More preferably, it refers to acid whey or cheese whey.

The milk is preferably bovine milk, more preferably bovine mature milk. Although bovine colostrum contains much more immunoglobulins than mature bovine milk, it is not an option to use whey from bovine colostrum for the production of an immunoglobulin-enriched whey protein-containing product. First of all, the composition of colostrum (e.g. its high concentration of whey proteins) is such that it tends to precipitate on the surface of heat exchangers and evaporators, causing problems in their cleaning and maintenance. In addition, the use of colostrum raises ethical issues, because it deprives newly born calves of the essential nutrition in the first few days of their life.

Within the content of the present invention, mature bovine milk is bovine milk other than colostrum. Colostrum is the milk in the first three days after calving. Colostrum has higher levels of fat, whey proteins (including Ig's), vitamins and minerals and lower levels of lactose and casein than mature bovine milk.

Acid whey is produced by subjecting the milk, preferably after skimming and pasteurization, to a casein precipitation process. This casein precipitation involves the addition of an acid to induce coagulation of casein, resulting in an acid casein fraction (a casein curd) and an acid whey fraction. The acid is preferably selected from HCl, H₂SO₄, and citric acid.

For example, 1 M H₂SO₄ may be added to skim milk, with sufficient stirring at 40-45° C., until a pH on the range 4.3-4.6 is reached. The acidified milk is stirred until curd formation is complete. The curd is then removed from the whey by means of, e.g., bag filtration or centrifugation.

Cheese whey is produced by subjecting the milk, preferably after skimming and pasteurization, to a cheese-making process The cheesemaking process preferably involves the addition of a coagulant and an acidifier, and allowing coagulation in order to obtain a casein-rich fraction (the cheese or cheese precursor) and a whey fraction. The target pH for the acidification is preferably in the range 4.8 to 5.7, more preferably 4.9 to 5.5. Suitable acidifiers include starter cultures (bacterial acidifiers) which convert lactose into lactic acid, acids, acidulants (such as for example Glucono Delta Lactone or GDL), and combinations of two or more of these. The most common starter cultures include thermophilic starters, typically starters from CSK, Chr. Hansen, or DuPont. Thermophilic starters by Chr. Hansen include frozen cultures STI-02, STI-03, STI-04, STI-06 and freeze-dried cultures STI-12, STI-13 and STI-14. Mesophilic starters may also be used.

Suitable coagulants are known in the art and include, for instance, calf rennet, fermentation-produced rennet and microbial rennet. Examples of calf rennet include Kalase produced by CSK and Naturen produced by Chr. Hansen. Examples of fermentation-produced rennet include Fromase by DSM and Milase by CSK. Examples of microbial rennets are Chy-Max by Chr. Hansen and Maxiren by DSM. Other coagulants include pepsin and various proteolytic enzymes of plant origin.

The casein-reduced milk will generally have a total IgG content in the range 2.5-10 wt %, preferably 2.5-5.0 wt %, based on total protein. The total protein content of the casein-reduced milk is determined by determining the total protein content and subtracting the non-protein nitrogen (NPN) and casein content from the total protein content; all determined by the well-known Kjeldahl method (conversion factor 6.38). The IgG content of the casein-reduced milk can be determined using the ELISA quantitation set as described by R. L. Valk-Weeber, T. Eshuis-de Ruiter, L. Dijkhuizen, and S. S. van Leeuwen, International Dairy Journal, Volume 110, November 2020, 104814).

The casein-reduced milk is subjected to cross-flow filtration using a membrane with a molecular weight cut-off (MWCO) of 500-1000 kDa, preferably 500-800 kDa, or a pore size of 50-100 nm, preferably 50-80 nm, thereby obtaining a permeate enriched in lactose, salts, α-lactalbumin, and β-lactoglobulin, and a retentate enriched—relative to total protein—in immunoglobulins.

This filtration step serves to increase the immunoglobulin concentration and purity of the stream in order to improve the binding capacity of the chromatography resin and the purity of the product of the next step.

Suitable types of cross-flow filtration membranes include spiral wound, ceramic, and hollow fibre membranes. Spiral wound membranes are preferred in view of their pricing and their suitability in large scale processes.

The membrane can be constructed form various polymer types, such as polysulfone (PS), (modified) polyethersulfone, polyvinylidene difluoride (PVDF), polyacrylonitrile (PAN), cellulose acetate (CA), and polypropylene (PP).

The trans-membrane pressure (TMP) across the membrane is preferably in the range of 0.25-1.5 bar, more preferably 0.25-1.0 bar, most preferably 0.25-0.5 bar.

The cross-flow filtration step is conducted at a temperature either in the range 10-15° C. or 50-55° C., preferably in the range 50-55° C.

The cross-flow is preferably in the range 0.1-0.3 m/s, more preferably 0.2-0.3 m/s, most preferably 0.25-0.3 m/s for spiral wound membranes; 5-7 m/s, most preferably 6-7 m/s for ceramic membranes; 1-3 m/s, more preferably 2-3 m/s, most preferably 2.5-3 m/s for hollow fiber membranes.

In order to further increase the immunoglobulin purity, diafiltration is desirably conducted.

The casein-reduced milk may be concentrated or diafiltrated at a fixed VCF.

The retentate preferably has an about neutral pH. That is, the pH is preferably in the range 6.0-8.0, more preferably 6.5-7.5. If the pH is not yet within this range, it can be regulated by the addition of acid or base.

The retentate is then subjected to a special type of chromatography, which we will herein be referred to as mixed mode chromatography or MultiModal chromatography. This type of chromatography involves a combination of hydrophobic and cationic interactions, also called hydrophobic charge-induction chromatography (HCIC). At neutral pH, the binding of proteins to the resin takes place by hydrophobic interactions; at low pH, the elution mechanism is based on electrostatic repulsion.

A preferred mixed mode resin is MEP HyperCel™, which is composed of a porous cellulose matrix linked with a 4-Mercapto-Ethyl-Pyridine (4-MEP) ligand. With a pKa of 4.8, the 4-MEP ligand is uncharged at neutral pH, so that the adsorption of immunoglobulins is mainly achieved by hydrophobic interactions.

Other suitable mixed mode resins are HEA Hypercel™, which contains an hexylamine ligand; Eshmuno® HCX chromatography resins, which contains strongly ionic sulfo groups, weakly ionic carboxyl groups, hydrophobically interacting phenyl groups, and hydrogen binding hydroxyl and amine groups; Nuvia cPrime, a hydrophobic cation exchange resin; and Capto™ MMC, a multimodal weak cation exchange resin.

The immunoglobulins are subsequently eluted from the resin using an aqueous solution, preferably having a pH of 2.0-7.0, more preferably 2.0-6.0, even more preferably 3.0-5.0, and most preferably 4.0-5.0. Preferably, a buffering solution is used to reach this pH. Any type of buffer may be used.

The resulting immunoglobulin-containing effluent can be submitted to further processing steps, if so desired. Examples of such processing steps are: demineralisation, e.g. by ultrafiltration and/or diafiltration; concentration, e.g. by evaporation or freeze concentration; and drying, e.g. spray-drying or freeze drying.

The whey protein-containing product according to the present invention and produced by the process according of claim 1 preferably comprises at least 40 wt %, preferably 50-80 wt % IgG, based on total protein. As mentioned above, the total protein content of this product is determined by the BCA method, as described in R. A. Brown et al., Analytical Biochemistry, Volume 180 (1), July 1989, 136-139, and the IgG content is determined using the RP-HPLC-based method as described by D. F. Elgar et al., Journal of Chromatogeraphy A, Volume 878, 2000, 183-196.

The whey protein-containing product resulting from the process of the present invention may be further concentrated, demineralized, and/or (spray) dried in order to make a powdered product.

The whey protein-containing product according to the invention is particularly suitable for use as an ingredient in the production of a nutritional composition, in particular formula milk. The formula milk is selected from the group of infant formulas, follow-up formulas and growing-up formulas. Accordingly, the invention further relates to a nutritional composition, typically a nutritional composition for a child, such as formula milk, in particular an infant formula, a follow-up formula, or a growing-up formula.

The nutritional composition, in particular the formula milk, can be prepared by combining the WPC with at least a lipid source, a carbohydrate source, vitamins, and minerals, and optionally further dairy and protein sources.

The lipid source may be any lipid or fat suitable for use in formula milk. Preferred fat sources include milk fat, safflower oil, egg yolk lipid, canola oil, olive oil, coconut oil, palm kernel oil, soybean oil, fish oil, palm oleic, high oleic sunflower oil, high oleic safflower oil, and microbial fermentation oil containing long-chain polyunsaturated fatty acids. In one embodiment, anhydrous milk fat is used. The lipid source may also be in the form of fractions derived from these oils such as palm olein, medium chain triglycerides, and esters of fatty acids such as arachidonic acid, linoleic acid, palmitic acid, stearic acid, docosahexaeonic acid, linolenic acid, oleic acid, lauric acid, capric acid, caprylic acid, caproic acid, and the like. Also small amounts may be added of oils containing high quantities of preformed arachidonic acid and docosahexaenoic acid such as fish oils or microbial oils. The fat source preferably has a ratio of n-6 to n-3 fatty acids of about 5:1 to about 15:1; for example about 8:1 to about 10:1. In a specific aspect, the infant formula comprises an oil mix comprising palmitic acid esterified to triacylglycerols, for example wherein the palmitic acid esterified in the sn-2 position of triacylglycerol is in an amount of from 20% to 60% by weight of total palmitic acid and palmitic acid esterified in the sn-1/sn-3 position of triacylglycerol is in an amount of from 40% to 80% by weight of total palmitic acid.

Examples of vitamins and minerals that are preferably present in formula milk are vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine. Minerals are usually added in salt form.

Examples of carbohydrates that are preferably present in formula milk are lactose, non-digestible oligosaccharides such as galacto-oligosaccharides (GOS) and/or fructo-oligosaccharides (FOS) and human milk oligosaccharides (HMOs).

If necessary, the nutritional composition may contain emulsifiers and stabilisers such as soy lecithin, citric acid esters of mono- and di-glycerides, and the like. It may also contain other substances which may have a beneficial effect such as lactoferrin, nucleotides, nucleosides, and the like.

### FIGURE

FIG. 1 illustrated the breakthrough of IgG during the chromatography steps of Examples 1-3. It show the percentage of IgG flowing through the column instead of binding to the resin. The lower the IgG levels flowing through the column, i.e. the lower the breakthrough, the higher the binding capacity.

### EXAMPLES

### Comparative Example 1

Cheese whey having a dry matter of 10 wt % was used as the starting material. Its pH was adjusted to 7.0. A known amount (2.5 l) of said cheese whey was loaded on a mixed mode chromatography column (MEP-Hypercel, 50 ml resin) with a flow rate of 4 batch volume per hour (BV/hr), which equalled 3.3 ml/min. After loading and subsequent rinsing, adsorbed proteins were stepwise eluted with (1) 0.050 mol/l MES (2-(N-morpholino)ethanesulfonic acid buffer of pH 6, (2) 0.05 mol/l sodium acetate buffer with pH 4.5, and (3) 0.1 mol/l glycine-HCl buffer of pH 2.7. The vast majority of the adsorbed proteins were released from the column when the resin was exposed to an acetate buffer (50 mM, pH=4,5) using a flowrate of 4 BV/hr.

The obtained fractions (Starting material, Breakthrough and Adsorbed) were analyzed for protein and IgG content using BCA and RP-HPLC. The obtained results are shown in Table 1.

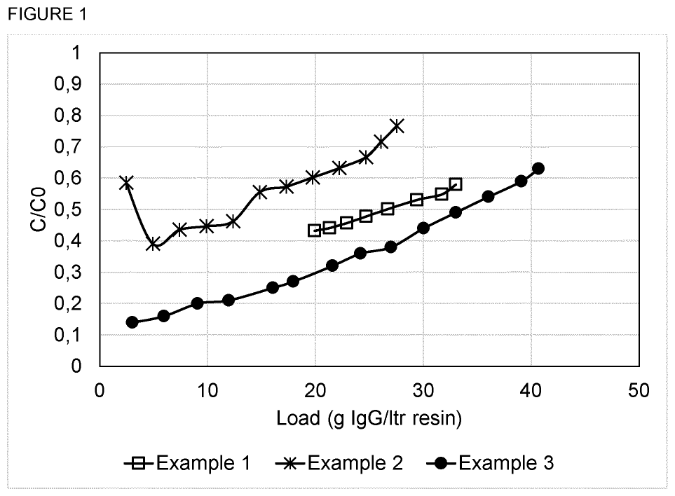

FIG. 1 shows the breakthrough curve, which illustrates the Ig concentration flowing through instead of binding to the resin.

### Comparative Example 2

Comparative Example 1 was repeated, except that the cheese whey, before being subjected to chromatography, was first diluted to a dry matter content of 6 wt % and then subjected to a crossfiltration process using a TAMI 8 kDa ceramic membrane. The applied crossflow was 15 l/min; the transmembrane pressure 1.5 bar. This resulted in a concentration of the retentate by a factor 4.

The pH of said retentate was adjusted to 7.0 and was then subjected to chromatography according to Example 1.

The obtained results are shown in Table 1; the breakthrough curve is shown in FIG. 1.

### Example 3

Comparative Example 2 was repeated, except that the crossflow filtration was conducted with a spiral wound 500 kD membrane. In total 400 kg of whey was circulated on the concentrate side. Filtration started at a crossflow of 80 l/min and a TMP of 0.5 bar. The product was concentrated to a final volume of approx. 33 l and the obtained concentrate was subjected to 1×33 l portions of diafiltration.

The pH of said retentate was adjusted to 7.0 and was then subjected to chromatography according to Example 1.

The obtained results are shown in Table 1; the breakthrough curve is shown in FIG. 1. It shows that the process of Example 3 leads to significantly reduced breakthrough—i.e. loss—of IgG, a higher IgG purity, and a higher yield compared to the processes of Examples 1 and 2. Hence, the binding capacity, IgG purity, and yield improved by performing, prior to the chromatography step, a crossflow filtration according to the present invention instead of no filtration of ultrafiltration with a 8 kDa membrane.

TABLE 1

| | Adsorbed (g/l resin) | | |
|---|---|---|---|
| | IgG | Protein (BCA) | Purity(%) |
| Example 1 | 14.5 | 21.4 | 68 |
| Example 2 | 9.6 | 15.1 | 64 |
| Example 3 | 26.5 | 27.6 | 96 |

1: Process for producing a whey protein-containing product enriched in immunoglobulins, said process comprising the steps of:
(i) cross-flow filtration of casein-reduced milk using a membrane with a molecular weight cut-off (MWCO) of 500-1000 kDa or a pore size of 50-100 nm, thereby obtaining a permeate enriched in lactose, salts, α-lactalbumin and β-lactoglobulin, and an UF retentate, and
(ii) subjecting said UF retentate to mixed mode chromatography, wherein immunoglobulins adhere to a resin and are subsequently eluted to form said whey protein-containing product enriched in immunoglobulins.

2: Process according to claim 1 wherein the casein-reduced milk is selected from acid whey, cheese whey, and native whey.

3: Process according to claim 1 wherein the whey protein-containing product formed in step (ii) is subjected from a concentration and/or drying step.

4: Process according to claim 1 wherein the whey protein-containing product comprises at least 40 wt %, based on total whey protein.

5: Process according to claim 1 wherein the retentate subjected to mixed mode chromatography has a pH in the range of 6.0-8.0.

6: Process according to claim 1 wherein the IgG content of the casein-reduced milk before subjecting to cross-flow filtration is in the range of 2.5-10 wt % based on total protein.

7: Process according to claim 1 wherein the membrane used in step (i) is a spiral wound membrane.

8: Whey protein-containing product obtainable by the process according to claim 1.

9: Whey protein-containing product according to claim 8 comprising at least 40 wt % based on total whey protein.

10: Nutritional composition comprising the whey protein-containing product according to claim 8.

11: Nutritional composition according to claim 10 wherein the nutritional composition is selected from infant formula, follow-up formula and growing-up milk

12: Process for producing a nutritional composition by combining the whey protein-containing product according to claim 8 with at least a fat source, a carbohydrate source, vitamins and minerals, and optionally further dairy and/or protein source(s).

13: Process according to claim 1, wherein the cross-flow filtration of casein-reduced milk uses a membrane with a molecular weight cut-off (MWCO) of 500-800 kDa or a pore size of 50-80 nm.

14: Process according to claim 2, wherein the casein-reduced milk is selected from acid whey and cheese whey.

15: Process according to claim 4, wherein the whey protein-containing product comprises 50-80 wt % IgG, based on total whey protein.

16: Process according to claim 5, wherein the retentate subjected to mixed mode chromatography has a pH in the range of 6.5-7.5.

17: Process according to claim 6, wherein the IgG content of the casein-reduced milk before subjecting to cross-flow filtration is in the range of 2.5-5.0 wt %, based on total protein.

18: Whey protein-containing product according to claim 9, comprising 50-80 wt % IgG, based on total whey protein.
